(19) United States
(12) Patent Application Publication
(10) Pub. No.: US 2015/0191857 A1
(43) Pub. Date: Jul. 9, 2015

(54) MANUFACTURING APPARATUS OF TAMPON AND MANUFACTURING METHOD OF TAMPON

(71) Applicant: UNI-CHARM CORPORATION, Shikokuchuo-shi (JP)

(72) Inventors: Katsuhiro SUZUKI, Fukushima (JP); Yoshitomo TAKASAKA, Fukushima (JP); Takahiro MUTO, Fukushima (JP)

(21) Appl. No.: 14/589,453

(22) Filed: Jan. 5, 2015

(30) Foreign Application Priority Data
Jan. 6, 2014 (JP) 2014-000203

(51) Int. Cl. D05B 47/06; A61F 13/34; A61F 13/20

(52) U.S. Cl. CPC D05B 47/06; A61F 13/2085; A61F 13/34

(57) ABSTRACT

A manufacturing apparatus of a tampon that has an absorber that absorbs bodily fluids and a removal string to remove the absorber from inside a vagina, the manufacturing apparatus of a tampon includes: a sewing machine that sews the absorber and the removal string together; and a defective sewing detecting portion that detects defective sewing between the absorber and the removal string.

### CROSS-REFERENCE TO RELATED APPLICATIONS

The present application claims priority upon Japanese Patent Application No. 2014-000203 filed on Jan. 6, 2014, which is herein incorporated by reference.

### BACKGROUND

1. Technical Field

The present invention relates to manufacturing apparatuses of tampons and manufacturing methods of tampons.

2. Related Art

A tampon which is a sanitary product generally has a bullet shaped absorber that absorbs bodily fluids such as menstrual blood and a removal string that extends out from a rear end of the absorber. Such a tampon is manufactured by sewing a removal string to a sheet absorber made by wrapping hydrophilic fiber such as rayon and cotton with a liquid permeable sheet, and then pressure molding the sheet absorber into a bullet shape (for example, refer to Japanese Patent Laid-open Application No. 2008-302115).

After the absorber has been inserted inside a vagina and has been used, a user of the tampon pulls the removal string to remove the absorber from inside the vagina. At that time, in the case that the removal string comes off from the absorber, the absorber cannot be removed from inside the vagina.

### SUMMARY

An advantage of some aspects of the present invention is that it is possible to suppress a tampon in which a removal string can easily come off from the absorber from being provided to a user.

An aspect of the invention is a manufacturing apparatus of a tampon that has an absorber that absorbs bodily fluids and a removal string to remove the absorber from inside a vagina, the manufacturing apparatus of a tampon including:
a sewing machine that sews the absorber and the removal string together; and
a defective sewing detecting portion that detects defective sewing between the absorber and the removal string.

Other features of the present invention will be made clear through the present specification with reference to the accompanying drawings.

### BRIEF DESCRIPTION OF THE DRAWINGS

For a more complete understanding of the present invention and the advantages thereof, reference is now made to the following description taken in conjunction with the accompanying drawings wherein:

FIG. 1 is an explanatory view of a tampon;

FIG. 2A to FIG. 2C are explanatory views of skipped stitches which is one example of defective sewing between a sheet absorber and a removal string;

FIG. 3A to FIG. 3C are explanatory views of other defective sewing;

FIG. 4A and FIG. 4B are explanatory views of a sewing unit of a manufacturing apparatus of tampons;

FIG. 5 is an explanatory view of the sewing unit of the manufacturing apparatus of tampons; and

FIG. 6A and FIG. 6B are graphs showing measurement results of an upper thread tension sensor.

### DESCRIPTION OF EXEMPLARY EMBODIMENTS

At least the following matters will become clear through the description of the present specification and the accompanying drawings.

A manufacturing apparatus of a tampon that has an absorber that absorbs bodily fluids and a removal string to remove the absorber from inside a vagina, the manufacturing apparatus of a tampon comprising:
a sewing machine that sews the absorber and the removal string together; and
a defective sewing detecting portion that detects detective sewing between the absorber and the removal string.

According to such a manufacturing apparatus of a tampon, the tampon in which an occurrence of defective sewing has been detected can be excluded, and the tampon in which the removal string can come off easily from the absorber can be suppressed from being provided to user.

A manufacturing apparatus of a tampon, wherein
the defective sewing detecting portion detects the defective sewing in a state where a tension is applied to the removal string along a longitudinal direction of the removal string.

According to such a manufacturing apparatus of a tampon, the removal string can be appropriately sewn to the absorber, and the defective sewing can be detected during sewing, and the defective sewing can be detected more accurately.

A manufacturing apparatus of a tampon, wherein
the defective sewing detecting portion defects the defective sewing based on a tension of a thread of the sewing machine that sews the absorber and the removal string together.

According to such a manufacturing apparatus of a tampon, the tampon in which the occurrence of the defective sewing has detected can be excluded, and the tampon in which the removal string can come off easily from the absorber can be suppressed from being provided to a user.

A manufacturing apparatus of a tampon, wherein
the defective sewing detecting portion detects the defective sewing by performing at least one of
   comparing a maximum value of the tension of the thread within a predetermined period and a first threshold value, and
   comparing the tension of the thread and a second threshold value lower than the first threshold value.

According to such a manufacturing apparatus of a tampon, a shipped stitch during sewing can be detected.

A manufacturing apparatus of a tampon, wherein
the defective sewing detecting portion detects the defective sewing, based on a tension of an upper thread that has been passed through a sewing needle that moves up and down, of the thread.

According to such a manufacturing apparatus of a tampon, the defective sewing can be detected more accurately.

A manufacturing apparatus of a tampon, wherein
the sewing machine has an upper thread tension regulator that regulates the tension of the upper thread and a thread take up lever that moves up and down in accordance with the up and down movement of the sewing needle, and
the detective sewing detecting portion detects the defective sewing based on the tension of the upper thread that is measured with a tension sensor provided between the upper thread tension regulator and the thread take up lever.

According to such a manufacturing apparatus of a tampon, the tension sensor can measure the tension of the upper thread stably without inhibiting the up and down movement of the thread take up lever, and can measure the tension of the upper thread in a position as near as possible to the position of occurrence of the detective sewing.

A manufacturing apparatus of a tampon, wherein
a tension regulating portion and a speed sensor are provided between the sewing machine and the supply source of the thread of the sewing machine that sews the absorber and the removal string together, the tension regulating portion regulating a tension of the thread fed from a supply source to be a predetermined value, the speed sensor measuring a speed in which the thread fed from the tension regulating portion is supplied to the sewing machine, and
the defective sewing defecting portion detects the defective sewing based on a measurement result of the speed sensor.

According to such a manufacturing apparatus of a tampon, the tampon in which an occurrence of defective sewing has been detected can be excluded, and the tampon in which the removal string can come off easily from the absorber can be suppressed from being provided to a user.

Further, a manufacturing method of a tampon that has an absorber that absorbs bodily fluids and a removal string to remove the absorber from inside a vagina, the manufacturing method of a tampon comprising:
sewing the absorber and the removal string together; and
detecting defective sewing between the absorber and the removal string.

According to such a manufacturing method of a tampon, the tampon in which an occurrence of defective sewing has been detected can be excluded, and the tampon in which the removal string cent come off easily from the absorber can be suppressed from being provided to a user.

### ===Configuration of a Tampon===

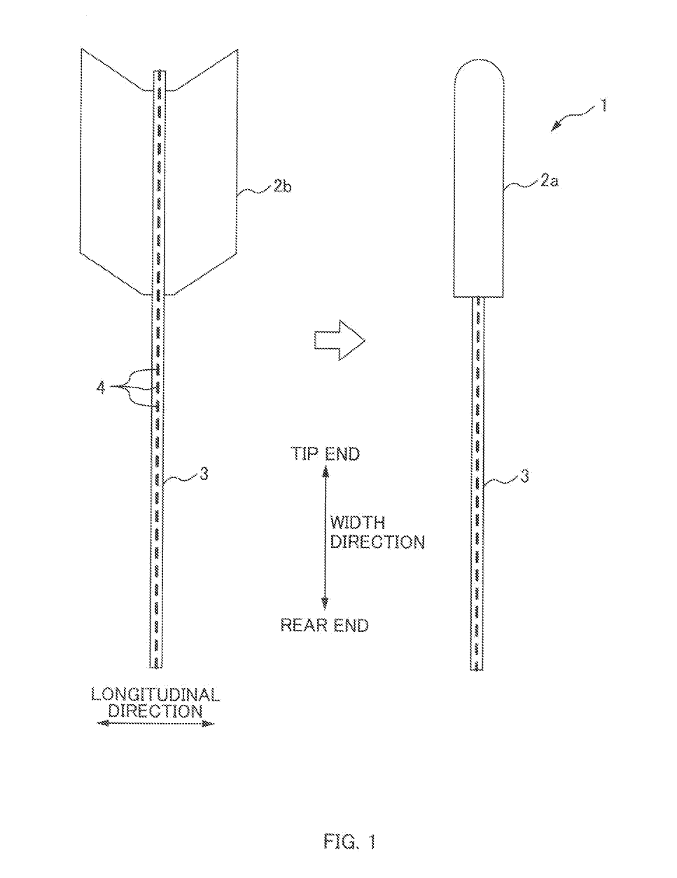

FIG. 1 is an explanatory view of a tampon 1. As shown in a right diagram in FIG. 1, the tampon 1 has a bullet shaped absorber 2a that is inserted inside a vagina and absorbs bodily fluids such as menstrual blood, and a removal string 3 (for example, a cotton twine) that is held when removing the absorber 2a from inside the vagina. For the purpose of explanation, in a longitudinal direction of the tampon 1, a side to be inserted inside the vagina is to be referred to as a tip end side, and the opposite side thereof is to be referred as a rear end side.

In a manufacturing method of the tampon 1, first a laminated body made by laminating absorbent sheets that absorb the bodily fluids (for example, a hydrophilic fiber sheet of rayon, cotton and the like) is covered on both surfaces with a liquid permeable sheet (for example, a polyester spun bonded nonwoven fabric), is cut out into a feather shape, and a sheet absorber 2b is manufactured. As shown in a left diagram in FIG. 1, the removal string 3 is laid along the longitudinal direction in a central portion in a width direction of one surface of the sheet absorber 2b, and the sheet absorber 2b and the removal string 3 are sewn and connected with a thread 4 (for example, a cotton thread). It should be noted that, in this embodiment, not only a section in which the sheet absorber 2b and the removal string 3 overlap, but also a section with only the removal string 3 that extends out from the rear end of the sheet absorber 2b is sewn with the thread 1 (an upper thread and a lower thread). It is not limited to the above however, and the section of only the removal string 3 does not have to be sewn.

Then, the sheet absorber 2b is compression molded into a ballet shape so that the surface side attached with the removal string 3 becomes the inside, and the absorber 2a is manufactured. Thus, the tampon 1 is shaped such that the removal string 3 is not exposed from the absorber 2a and extends out from a rear end of the absorber 2a. Then, the tampon 1 that has been manufactured is housed inside an applicator that is for inserting the tampon 1 inside the vagina, is wrapped, and provided to a user.

===Defective Sewing between Absorber and Removal String===

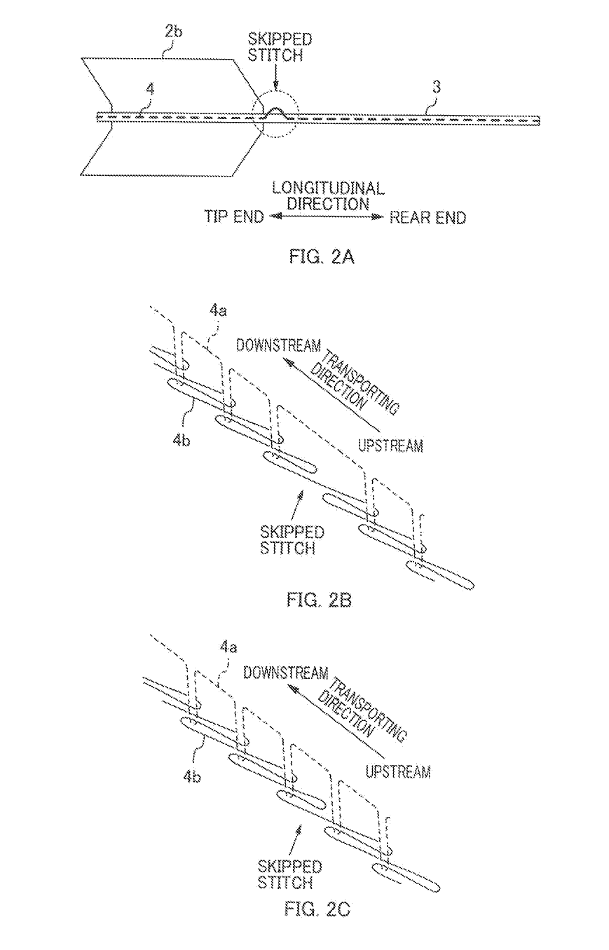

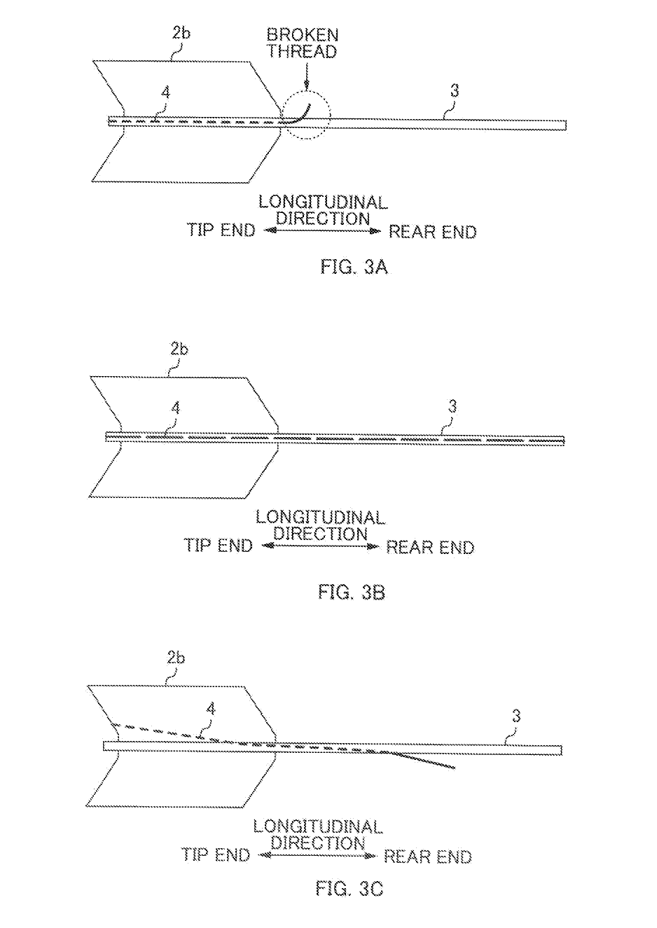

FIG. 2A to FIG. 2C are explanatory views of cases of skipped stitches which is one example of defective sewing between the sheet absorber 2b and the removal string 3. FIG. 3A to FIG. 3C are explanatory views of other cases of defective sewing. In this embodiment, the sheet absorber 2b and the removal string 3 are sewn with a sewing machine that does double chain stitching, and there is a case where detective sewing occurs during sewing with the sewing machine. It should be noted that, the sewing method of the sheet absorber 2b and the removal string 3 is not limited to the double chain stitching, and other methods of sewing, for example, lock stitching may to used.

As an example of defective sewing, as shown in FIG. 2A, there is the “skipped stitch” in which a seam is skipped. As shown in FIG. 2B, a skipped stitch occurs because a looper (not shown) through which a lower thread 1b has been passed cannot pick up a loop of an upper thread 4a that has been passed through a sewing needle (not shown), or on the other hand, as shown in FIG. 2C, because the sewing needle cannot pick up a loop of the lower thread 4b. In particular, during sewing of the tampon 1, a resistance of the sewing needle changes in a boundary between a section in which the sheet absorber 2b and the removal string 3 overlap and a section with only the removal string 3, thus the skipped stitch easily occurs. When the skipped stitch occurs, the seam easily becomes loose, and the removal string 3 easily comes off from the absorber 2a of the tampon 1. It should be noted that, in double chain stitching, the lower thread 4b entwines twice with the upper thread 4a, and as shown in FIG. 2B and FIG. 2C, it becomes easier to loosen in the downstream side (a side in which sewing is started) in the transporting direction, and it becomes harder to loosen in the upstream side in the transporting direction (a side in which the sewing ends).

As other examples of defective sewing, as shown in FIG. 3A, there is a case where the thread 4 breaks daring sewing of the sheet absorber 2b and the removal string 3. In this case, the seam comes apart from the point where the thread 4 has broken, and the length of the seam is short and the strength of the seam is weak, thus the removal string 3 easily comes off from the absorber 2a. As shown in FIG. 3B, there are other cases where the seam tightness is bad, or the seam is rough. Also in these cases, the sewing strength is weak and the removal string 3 easily comes off from the absorber 2a. As shown in FIG. 3C, there are cases where the sewing position becomes misaligned, and only the sheet absorber 2b is sewn, or the stitch is not sewn. Also in this case, the strength of the seam is weak, and the removal string 3 easily comes off from the absorber 2a. Further, as shown in FIG. 3C, there are cases where the sewing position becomes misaligned, and only the sheet absorber 2b is sewn, or empty sewing occurs. Also in those cases, the strength of the seam is weak, and the removal string 3 easily comes off from the absorber 2a.

As described above, when defective sewing occurs during sewing of the sheet absorber 2b and the removal string 3, and the removal string 3 is in a state where it can easily come off from the absorber 2a, the removal string 3 come off from the absorber 2a when the user of the tampon 1 pulls the removal string 3 to remove the absorber 2a from inside the vagina. In that case, the absorber 2a cannot be removed from inside the vagina. Thus, with the manufacturing apparatus and the manufacturing method of the tampon 1 of this embodiment, defective sewing between the absorber 2a (sheet absorber 2b) and the removal string 3 is detected. As a result, the tampon 1 that has been detected to have defective sewing can be excluded, and the tampon 1 in which the removal string 3 easily comes off from the absorber 2a can be suppressed from being provided to a user.

===Detection of Defective Sewing between Absorber and Removal String===

### <<Sewing Unit>>

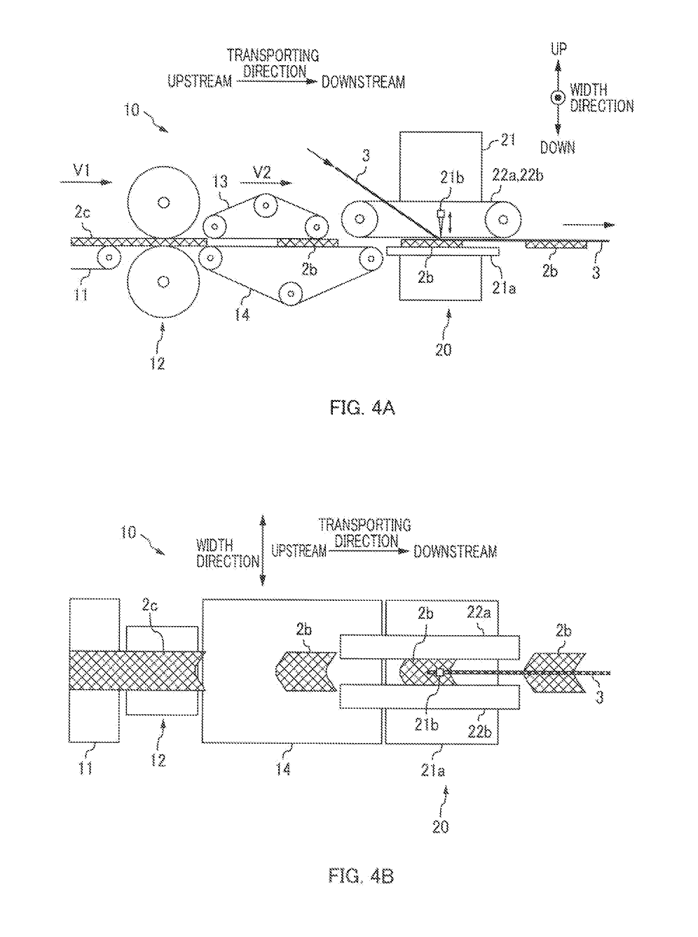

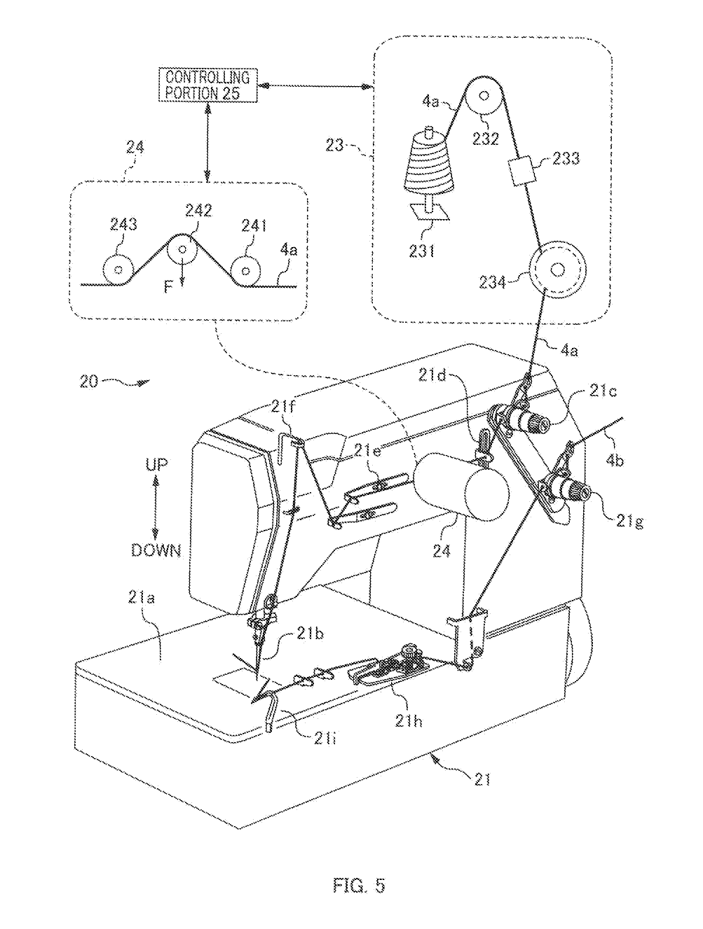

FIG. 4A, FIG. 4B, and FIG. 5 are explanatory views of a sewing unit 20 of manufacturing apparatus 10 of the tampon 1. It should be noted that, in the figure, a longitudinal direction of the sheet absorber to (tampon 1) is a transporting direction of materials, and a thickness direction of the sheet absorber 2b is an up-down direction. Further, FIG. 4a is a sectional view in which a width direction of the sheet absorber 2b is a normal direction, and FIG. 4B is a plan view in which an up-down direction is a normal direction.

As described above, in the manufacturing method of the tampon 1, first a continuous body 2c of the sheet absorbers, which is a laminated body laminated with absorbent sheets that is covered on both surfaces with a liquid permeable sheet, is manufactured. As shown in FIG. 4A and FIG. 4B, a shape in a planar view of the continuous body 2c of the sheet absorber is a rectangular shape, and the continuous body 2c of the sheet absorber is transported to a rotary die cutter 12 with a first belt conveyor 11, along a transporting direction in which the continuous body continues. The rotary die cutter 22 is a pair of rollers that are opposed to each other in the up-down direction, and a cutting blade is provided in a peripheral surface of one of the rollers. Thus, the continuous body 2c of the sheet absorber that has passed through between the pair of rollers configuring the rotary die cutter 12 is sandwiched between the cutting blade of one of the rollers and the peripheral surface of the other roller, and thus cut into a feather shaped sheet absorber 2b. The cut sheet absorber 2b is sandwiched between a second belt conveyor 13 positioned to an upper side and a third belt conveyor 14 positioned to a lower side, and transported to the sewing unit 20.

It should be noted that, the sewing unit 20 continuously sews one removal string 3, with respect to the sheet absorbers 2b that are transported in the transporting direction with intervals between the sheet absorbers without stopping. Thus, the interval in the transporting direction between the sheet absorbers 2b transported to the sewing unit 20 needs to be made equal to or longer than the length of the removal string 3 that extends out from the rear end of the absorber 2a of the tampon 1. Then, a transporting speed V2 of a second belt conveyor 13 and a third belt conveyor 14 in the downstream side in the transporting direction is made faster than a transporting speed V1 of a first belt conveyor 11 in an upstream side in the transporting direction than the rotary die cutter 12. In this way, the sheet absorber 2b that has been cut can be separated in the transporting direction.

The sewing unit 20 includes a sewing machine 21, a pair of press belt conveyors 22a and 22b, a thread supplying portion 23, an upper thread tension sensor 24, and a controlling portion 25, and the thread supplying portion 23, the upper thread tension sensor 24, and the controlling portion 25 correspond to a defective sewing detecting portion. The sheet absorber 2b that is transported with the second and the third belt conveyors 13 and 14 is sandwiched between a downstream side end portion of the third belt conveyor 14 and the pair of the press belt conveyors 22a and 22b, and transported to a workbench 21a of the sewing machine 21. On the workbench 21a, the sheet absorber 2b is transported to the downstream side in the transporting direction at a constant speed with the pair of the press belt conveyors 22a and 22b.

As shown in FIG. 4B, the pair of press belt conveyors 22a and 22b is arranged with an interval in between in the width direction. Thus, a central portion of the sheet absorber 2b in the width direction is exposed and can be opposed with the sewing needle 21b of the sewing machine 21. Then, the removal string 3 is supplied along the transporting direction above the central portion in the width direction of the sheet absorber 2b that is exposed from between the pair of the press belt conveyors 22a and 22b, and the sheet absorber 2b and the removal string 3 are sewn with the sewing machine 21.

It should be noted that, one removal string 3 is sewn continuously with respect to the plurality of the sheet absorbers 2b that are transported with intervals in between the sheet absorbers in the transporting direction. Then, the removal string 3 is sewn to the sheet absorber 1b and transported to the downstream side in the transporting direction, thus is fed from a supply source (not shown) of the removal string 3, and supplied onto the sheet absorber 2b. Then, the sheet absorber 2b and the removal string 3 are sewn in a state where the removal string 3 is applied with tension along the longitudinal direction of the removal string 3, thus the removal string 3 can be appropriately sewn along the longitudinal direction of the sheet absorber 2b (tampon 1).

The thread supplying portion 23 includes a thread supplying portion 23 for an upper thread and a thread supplying portion for a lower thread (not shown), and since they have the same configuration, the thread supplying portion 23 for the upper thread is explained as an example. The thread supplying portion 23 tor the upper thread is for supplying the upper thread 4a to the sewing machine 21, and as shown in FIG. 5, includes a stand mechanism 231 that holds the upper thread 4a wound in a cone shape so that the upper thread can be fed, an upstream side tension sensor 233 that measures a tension of the upper thread 4a, a feeding roller 232 positioned between the stand mechanism 231 and the upstream side tension sensor 233, and a speed sensor 234 positioned to the downstream side of the upstream side tension sensor 233. Then, the thread supplying portion 23 for the upper thread makes the tension supplied to the sewing machine 21 constant. For that reason, according to the tension of the upper thread 4a that has been measured with the upstream side tension sensor 233, the controlling portion 23 controls the drive of a motor (not shown) which is a driving source of a feeding roller 232, and adjusts the feeding amount of the upper thread 4a from the stand mechanism 231.

Further, the speed of the upper thread 4a supplied to the sewing machine 21, namely, the amount (length) of the upper thread 4a supplied to the sewing machine 21 per unit time is measured with the speed sensor 234. It should be noted that, by providing the speed sensor 234 more to the downstream side than the upstream side tension sensor 233, and by the speed sensor 234 measuring the speed of the upper thread 4a that is regulated so that the tension is constant, the speed of the upper thread 4a can be measured more accurately. Further, as the speed sensor 234, there is, for example, a rotary encoder having a roller which is wrapped around with the upper thread 4a, a rotary scale that rotates integrally with the roller and is formed with slits at constant intervals in the circumferential direction, a light-emitting device and a light receiving device. With the rotary encoder, the rotation angle per unit time is obtained by whether or not the light receiving device has received the light from the light-emitting device that has passed through the slits. Thus, according to the rotation angle per such unit time, the speed of the upper thread 4a supplied to the sewing machine 21 can be measured. The speed sensor 234 is not limited to the rotary encoder however, as long as the device can measure the speed of the upper thread 4a.

The sewing machine 21 is for sewing together the sheet absorber 2a and the removal string 3, and the sewing machine is a general-purpose industrial sewing machine that can perform double loop stitching. As shown in FIG. 5, the upper thread 4a supplied from the thread supplying portion 23 for the upper thread is passed through the upper thread tension regulator 21c that regulates the tension of the upper thread 4a, a first thread guide 21d, an upper thread tension sensor 24, a second thread guide 21e, and a thread take up lever 21f that moves up and down according to the up and down movement of the sewing needle 21b, and through the sewing needle 21b. On the other hand, a lower thread 4b supplied from the thread supplying portion for the lower thread (not shown) is passed through a lower thread regulator 21g that regulates the tension of the lower thread 4b, a lower thread feeding cam 21h that adjusts the feeding amount of the lower thread 4b to a looper 21i, and is passed through the looper 21i that reciprocally moves in a direction intersecting the transporting direction. Then, with the up and down movement of the sewing needle 21b that penetrates through the sheet absorber 2b that is transported with the pair of press belt conveyors 22a, 22b and the removal string 3 and the reciprocating movement of the looper 21i, the entwining movement of the upper thread 4a and the lower 4b is repeated, and the sheet absorber 2b and the removal string 3 are sewn together. It should be noted that, in FIG. 5, the lower thread feeding cam 21h, the looper 21i and the like are virtually shown, and the pair of press belt conveyors 22a, 22b are omitted. Further, the up and down movement of the sewing needle 21b and the thread take up lever 21f are movements along the direction intersecting the surface of the workbench 21a, and is not limited to a movement along the up and down direction of the manufacturing apparatus 10 of the tampon 1.

Further, with the manufacturing apparatus of the tampon 1, due to problems such as in view of design, and hygiene, the supply source (stand mechanism 231) of the upper thread 4a and the lower thread 4b are in some cases arranged separate from the sewing machine 21. Thus, with only the upper thread tension regulator 21c and the lower thread regulator 21g that are provided to the sewing machine 21, which are operated manually by regulator knobs, it is difficult to regulate the tension of the upper thread 4a and the lower thread 4b. Thus, the feeding roller 232 and the upstream side tension sensor 233 are provided in between the supply sources of the upper thread 4a and the lower thread 4b and the sewing machine 21, and the tension supplied to the sewing machine 21 is preferably kept constant. In that way, the seam tightness can be made stable, and the defective sewing due to poor tightening of the seam as shown in the above described FIG. 3B can be prevented. As a result, the tampon 1 in which the removal string 3 easily comes off from the absorber 2a can be inhibited from being provided to the user.

The upper thread tension sensor 24 is provided to the sewing machine 21 later, and measures the tension of the upper thread 4a between the upper thread tension regulator 21c and the thread take up lever 21f. As a specific example of the upper thread tension sensor 24, as shown in FIG. 5, there is one in which the upper thread 4a is wrapped around the first roller 241, the second roller 242, and the third roller 243 in that order, and a strain gage is attached to a shaft that supports the second roller 242 in a cantilever state. With the upper thread tension sensor 24 configured as above, a downward force F applied to the second roller 242, namely, a combined force F of a tension of the upper thread 4a between the first, second rollers 241, 242 and a tension of the upper thread 4a between the second, third rollers 242, 243 is obtained based on the displacement amount of the strain gage, thus the tension of the upper thread 4a can be measured. The upper thread tension sensor 24 is not limited to the above configuration however, as long as the sensor can measure the tension of the upper thread 4a.

The sheet absorber 2b and the removal string 3 that are sewn together with the sewing unit 20 having the above configuration are thereafter transported to a step in which the removal string 3 is cut for each product of the tampon 1, and to a step of pressure molding the sheet absorber 2b into a bullet shape.

It should be noted that, the thread 4 also breaks when cutting the removal string 3 for each product of the tampon 1, but in the case of the double loop stitching, the stitch easily comes undone to the downstream side (a side where the sewing is started) in the transporting direction, and becomes harder to come undone to the upstream side (a side where the sewing is ended) in the transporting direction. Thus, as shown in FIG. 4B, when the tip end in the longitudinal direction of the sheet absorber 2b that is feather shaped (the side that is depressed in the central portion in the width direction) is faced to the downstream side in the transporting direction, the thread 4 becomes harder to come undone from the tip end to the rear end in the longitudinal direction of the tampon 1. In other words, the sewn section in which the sheet absorber 2b and the removal string 3 has been sewn together can be made harder to come undone, and as a result, the tampon 1 in which the removal string 3 easily comes off from the absorber 2a can be suppressed from being provided to the user.

### <<Detection of Defective Sewing>>

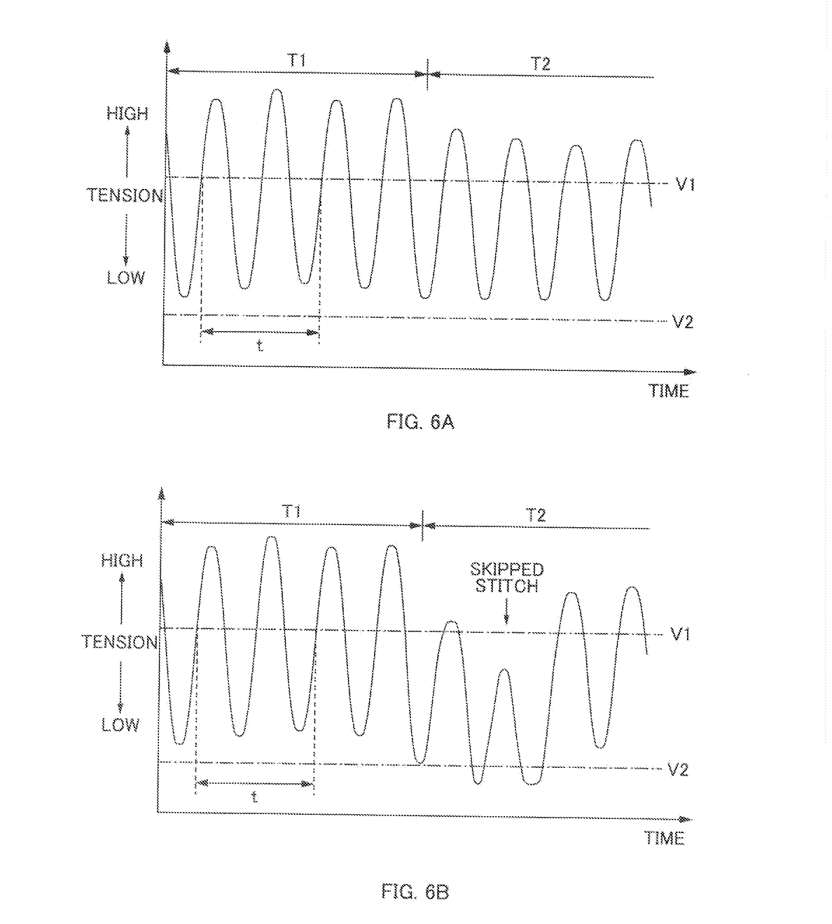

FIG. 6A and FIG. 6B are graphs showing measurement results of the upper thread tension sensor 24. A horizontal axis of the graph shows time, and a vertical axis shows tension of the upper thread 4a. FIG. 6A is the measurement result in the case where defective sewing did not occur during sewing together of the sheet absorber 2b and the removal string 3 with the sewing machine 21, and FIG. 6B is the measurement result in the case where a skipped stitch (FIG. 2) occurred. As shown in FIG. 6A, during sewing with the sewing machine 21, the tension of the upper thread 4a changes periodically up and down. This one period of change in tension of the upper thread 4a corresponds to one up and down movement of the sewing needle 31b. Then, when the seeing needle 21b moves up and the looper 21i picks up a loop of the upper thread 4a, and the upper thread 4a and the lower thread 4b entwine, the tension of the upper thread 4a becomes a maximum value. Further, in this embodiment, there are a first period T1 in which a section where the sheet absorber 2b and the removal string 3 overlap and a second period T2 in which the section of only the removal string 3 is sewn, and in the second period T2, when compared to the first period T1, a maximum value of the tension of the upper thread 4a becomes lower, and the change in tension (amplitude) of the upper thread 4a also becomes smaller. It should be noted that, the same measurement results are obtained regarding the tension of the thread, not only in the case where double loop stitching is performed, but also in the case where other sewing methods are performed in which the upper thread 4a and the lower thread 4b entwine.

Then, as shown in FIG. 6B, when the skipped stitch occurs during sewing the sheet absorber 2a and the removal string 3 together, the maximum value of the tension of the upper thread 4a within one period decreases because the upper thread 4a and the lower thread 4b do not entwine. Further, when the shipped stitch occurs, the upper thread 4a and the lower thread 4b do not entwine, and the upper thread 4a is not used and thus loosens, thus a minimum value of tension of the upper thread 4a within one period also decreases.

Then, in this embodiment, the controlling portion 25 having the sewing unit 20 obtains the measurement result of the upper thread tension sensor 24, during sewing of the sheen absorber 2b said the removal string 3 (T1, T2), and detects the skipped stitch (defective sewing), based on the tension of the upper thread 4a (thread) of the sewing machine 21 that sews together the sheet absorber 2b and the removal string 3.

Specifically, the controlling portion 25 compares the maximum value of the tension of the upper thread 4a within a predetermined period t and a first threshold value V1, and further compares the tension of the upper thread 4a and a second threshold value V2 that is lower than the first threshold value V1. Then, as shown in FIG. 6B, the controlling portion 25 acknowledges that the skipped stitch has occurred in the case where at least one of the case where the maximum value of the tension of the upper thread 4a within the predetermined period t becomes smaller than (or equal to or smaller than) the first threshold value V1 and the case where the tension of the upper thread 4a becomes smaller than (or equal to or smaller than) the second threshold value V2 has occurred. In this way, by detecting the skipped stitch during sewing of the sheet absorber 2b and the removal string 3 together, the tampon 1 in which the skipped stitch has occurred can be excluded, and the tampon 1 in which the removal string 3 can easily come off from the absorber 2a due to defective sewing between the absorber 2a and the removal string 3 can be suppressed from being provided to the user.

It should be noted that, contrary to the comparison with the second threshold value V2, in which occurrence of the skipped stitch is detected in the case where the tension of the upper thread 4a becomes lower than the second threshold value V2 for even one moment, in the comparison with the first threshold value V1, the maximum value of the tension of the upper thread 4a and the first threshold value V1 are compared within the predetermined period t. When this predetermined period t is set to be lower than one period in the tension change of the upper thread 4a, a timing in which the tension of the upper thread 4a becomes a maximum value is not included within that period t, and there is a possibility of a faulty detection of occurrence of the skipped stitch when the skipped stitch has not occurred. Thus, the predetermined period t is preferably set to at least a length equal to or greater than one period in the periodical change of the tension of the upper thread 4a. On the other hand, when the predetermined period t is set too long, it becomes difficult to specify a position where the period has occurred in which the maximum value of the tension of the upper thread 4a is smaller than the first threshold value V1. Thus, there is a possibility that the tampon in which the skipped stitch has occurred will not be accurately detected. Then, in this embodiment, the predetermined period, t is the length of approximately two periods in the periodical change of the tension of the upper thread 4a.

The detecting method of the skipped stitch with the tension of the upper thread 4a is not limited to the above method, and for example, a third threshold value which is a little higher than the second threshold value V2may be set, and in the case where the tension of the upper thread 4a becomes smaller than the third threshold value two times in a row, the skipped stitch may be detected. Further, in the case where both the case that the maximum value of the tension of the upper thread 4a within the predetermined period t becomes smaller than the first threshold value V1 and the case that the tension of the upper thread 4a becomes smaller than the second threshold value V2, the skipped stitch may be detected. Further, as described above, compared to the first period T1 in which the overlapping section of the sheet absorber 2b and the removal string 3 are sewn together, the second period T2 in which the section of only the removal string 3 is sewn becomes lower than the maximum value of the tension of the upper thread 4a, and the first period T1 and the second period T2 may also detect the skipped stitch with the same threshold values V1, V2. In that way, the detection of the skipped stitch with the controlling portion 23 may be easily performed.

Further, in this embodiment, as shown in FIG. 4B, the tip end of the tampon 1 is transported facing the downstream side in the transporting direction, and the thread 4 is also cut when cutting the removal string 3 of each product of the tampon 1. Thus, even if the skipped stitch occurs before transition from the second period T2 in which the section of only the removal string 3 is sewn to the first period T1 in which the overlapping section of the sheet absorber 2b and the removal string 3 are sewn, such skipped stitch section is cut when cutting the removal string 3. Then, the controlling portion 25 may determine that there is no problem even when the skipped stitch occurs before the transition from the second period T2 to the first period T1. In this way, according to the sewing position of the sheet absorber 2b and the removal string 3, the skipped stitch can be determined as to whether or not it will influence how easily the removal string 3 will come off from the absorber 2a. In this way, the tampon in which the removal string 3 can easily come off from the absorber 2a can be suppressed from being provided to the user, and the amount of waste of the material can be lessened.

Further, FIG. 6 shows the measurement result of the tension of the upper thread 4a, and the tension of the lower thread 4b similarly changes in periods up and down in accordance with the reciprocating movement of the looper 21i. In actuality however, when sewing together the sheet absorber 2b and the removal string 3 by double loop stitching with the sewing machine 21, as a result of measuring the tension change (amplitude) of the upper thread 4a and the tension change (amplitude) of the lower thread 4b, a result that the tension change of the upper thread 4a is bigger compared to the tension change of the lower thread 4b was obtained. This principle has not been accurately clarified, but it is considered that points such as that a supply path of the upper thread 4a and a supply path of the lower path 4b are different in the sewing machine 21 and that although the lower thread 4b is taken by the sewing needle 21b the upper thread 4b is taken by the looper 21i influence the above.

Then, when the tension change of the upper thread 4a is larger compared to the tension change of the lower thread 4b, the difference between the tension during occurrence of the skipped stitch and the tension during the normal time is also larger with the upper thread 4a than the lower thread 4b. Then, in this embodiment, the tension of the lower thread 4b is not measured, and the controlling portion 25 detects the shipped stitch based on the tension of the upper thread 4a. In this way, the skipped stitch can be accurately detected, while lessening the sensor that measures the tension of the lower thread 4b, and making the process of the controlling portion 25 easier. It is not limited to the above however, and the skipped stitch may be detected by providing the sensor that measures the tension of the lower thread 4b to the sewing machine 21, and based on both the tension of the upper thread 4a and the tension of the lower thread 4b, or the shipped stitch may be detected based on only the tension of the lower thread 4b.

Further, the controlling portion 25 detects the skipped stitch based on the tension of the upper thread 4a measured with the upper thread tension sensor 24 provided between the upper thread tension regulator 21c and the thread take up lever 21f of the sewing machine 21. In this way, by providing the upper thread tension sensor 24 between the upper thread tension regulator 21c and the thread take up lever 21f, the upper thread tension sensor 24 can measure the tension of the upper thread 4a stably, without inhibiting the up and down movement of the thread take up lever 21f. Further, since the tension of the upper thread 4a can be measured in the sewing portion, namely, a position as close as possible to the position in which the defective sewing occurred, the skipped stitch can be detected even more accurately. It is not limited to this however, and for example, the upper thread tension sensor 24 may be provided in a position near to the upstream side than the upper thread tension regulator 21c.

Up to here, detection of the skipped stitch based on the tension of the upper thread 4a has been explained, and defective sewing other than the skipped stitch can be detected based on the tension of the upper thread 4a. For example, as shown in FIG. 3B, in the ease that seam tightness is poor, the tension of the upper thread 4a decreases overall. Then, for example, in the case that the seam tightness is poor, and an average value of maximum values (or minimum values) of the tension of the upper thread 4a within one period is equal to or lower than the threshold value, the controlling portion 25 can detect that defective sewing has occurred. Further, as shown in FIG. 3C, in the case chat the sewing position becomes misaligned, and empty sewing occurs, the tension of the upper thread 4a gradually decreases, and thus the controlling portion 25 can detect the occurrence of the defective sewing. In this way by detecting defective sewing other than the skipped stitch, the tampon 1 in which the removal string 3 can easily come off from the absorber 2a can be more certainly suppressed from being provided to the user.

With the sewing unit 20 of this embodiment, as described above, after the tension of the upper thread 4a and the lower thread 4b fed from each supply source is regulated to be predetermined values, with the feeding roller 232, the upstream side tension sensor 233, and the controlling portion 25, in between each supply source (stand mechanism 231) of the upper thread 4a and the lower thread 4b, the speed at which the upper thread 4a and the lower thread 4b are supplied to the sewing machine 21 is measured with the speed sensor 234. It should be noted that, the feeding roller 232, the upstream side tension sensor 233, and the controlling portion 25 combined together correspond to a tension regulating portion. Then, during sewing of the sheet absorber 2b and the removal string 3 (T1, T2), the controlling portion 25 obtains the measurement result of the speed sensor 234, and detects defective sewing based on such measurement result.

As shown in FIG. 2, for example, in the case that the shipped stitch occurs, since the upper thread 4a and the lower thread 4b do not entwine, the upper thread 4a and the lower thread 4b are not used, and the supply speed, of the upper thread 4a and the lower thread 4b to the sewing machine 21 decreases. Further, as shown in FIG. 3A, in the case that the upper thread 4a and the lower thread 4b break during sewing, the upper thread 4a and the lower thread 4b are not supplied to the sewing machine 21, and the supplying speed of the upper thread 4a and the lower thread 4b decreases. Further, as shown in FIG. 3B, in the case that the seam tightening is poor, the upper thread 4a and the lower unread 4b are used a little, and the supplying speed of the upper thread 4a and the lower thread 4b decreases. Further, as shown in FIG. 3C, in the case that the sewing position is misaligned and empty sewing occurs, the use of the upper thread 4a and the lower thread 4b decreases, and the supplying speed of the upper thread 4a and the lower thread 4b decreases. Thus, in the case that the supplying speed of the upper thread 4a and the lower thread 4b to the sewing machine 21 that is measured with the speed sensor 234 becomes smaller then a threshold value, the controlling portion 23 can detect that defective sewing has occurred. In this way, not only is defective sewing detected based on the measurement result of the upper thread tension sensor 24, but also defective sewing is detected based on the measurement result of the speed sensor 234, and thus defective sewing can be detected more accurately, and the tampon 1 in which the removal string 3 can come off easily from the absorber 2a can be more certainly suppressed from being provided to the user.

In this way, in this embodiment, based on the measurement result of the upper thread tension sensor 24 and the speed sensor 234, the defective sewing during sewing of the sheet absorber 2b and the removal string 3 is defected. In other words, the defective sewing is detected along the longitudinal direction of the removal string 3, and in a state with tension applied to the removal string 3. In this way, the removal string 3 is appropriately sewn along the longitudinal direction of the sheet absorber 2b, and the defective sewing between the sheet absorber 2b and the removal string 3 can be quickly detected. Thus, the manufacturing can be quickly stopped when the defective sewing is detected, and thus the waste amount of the material due to defective sewing can be lessened.

### Modified Example

In the above embodiment, the upper thread tension sensor 24 is provided to the sewing machine 21, and the defective sewing is detected based on the tension of the upper thread 4a, but it is not limited to this. For example, a defective sewing detecting portion having a camera (for example, a CCD camera) and an image processing portion may be provided to the downstream side of the sewing unit 20, and based on an image data taken with the camera of the sheet absorber 2b and the removal string 3, the image processing portion may detect the defective sewing.

For example, in the case that the skipped stitch occurs in the part of only the removal string 3, as shown in FIG. 2A, the thread 4 rises up from the removal string 3. Then, in contrast to the color of the removal string 3 and the thread 4 (generally white), the belt conveyor and the like to be the background is made to be a dark color (for example, black), and the camera takes a picture of each product of the tampon 1 after the sheet absorber 2b and the removal string 3 have been sewn together. Then, in such image data, from a brightness difference between the part corresponding to the removal string 3 and the thread 4 and the part corresponding to the background, the image processing portion extracts an edge of the removal string 3 and the thread 4. In this way, in the case that the skipped stitch has occurred as shown in FIG. 2A, an edge width of the removal string 3 and the thread 4 become partially large, thus the occurrence of the skipped stitch can be detected. It should be noted that, in the case that the skipped stitch has occurred, the thread 4 is not limited to rising up in the width direction as shown in FIG. 2A, and there is also a case where the thread 4 rises up in the up down direction. Thus, for example, the image is preferably taken from at least two or more directions, such as a planar view image taken in the up down direction and a side view image taken in the width direction. For that purpose, a plurality of cameras may be provided, and the cameras may be rotated around the sheet absorber 2b and the removal string 3.

Further by making the color of the thread 4 different with respect to the color of the sheet absorber 2b and the removal string 3 (generally white), a part corresponding to the thread 4 can be extracted, from the image data taken with the camera. In particular, the color of the irradiating light and the color of the thread 4 are preferably set so that the irradiating light that is irradiated to the sheet absorber 2b and the removal string 3 from the same side as the camera can be absorbed by the thread 4. In this way, a difference between a reflecting light amount from the sheet absorber 2b and the removal string 3 and the reflecting light amount from the thread 4 becomes large, and the part corresponding to the thread 4 can be more accurately extracted from the image data. With the data of the thread 4 extracted in this way, for example, in the case that the seam is long, occurrence of the skipped stitch (FIG. 2) and the poor seam tightness (FIG. 3B) can be detected, and in the case where the seam is cut midway, the occurrence of the thread breaking (FIG. 3A) can be detected, and in the case that the angle of the direction in which the seam extends is inclined with respect to the longitudinal direction of the tampon 1, the occurrence of the misalignment of the sewing position (FIG. 3C) can be detected.

In this way, in the case of detecting defective sewing based on the image data taken with the camera, the defective sewing is preferably detected long the longitudinal direction of the removal string 3, in a state of applying tension to the removal string 3. In that way, the thread 4 (FIG. 2A) that has risen from the removal string 3 due to the skipped stitch becomes easier to detect, and the state of the thread 4 (seam) can be more accurately grasped from the image data, thus the detective sewing can be detected more accurately. Further, in the case that the defective sewing is detected based on the image data taken with the camera, the defective sewing may be detected after cutting the removal string 3 for each product of the tampon 1. In this case, by sucking and the like the removal string 3 with air, tension can be applied to the removal string 3.

Further, as described above, the defective sewing can be detected based on the measurement result of the speed sensor 234. Thus, it is not limited to detecting the defective sewing based on the tension of the thread 4, and the defective sewing may be detected based on only the measurement result of the speed sensor 234. Further, it is not limited to providing the sensor that measures the tension of the thread 4 to detect defective sewing to the sewing machine 21, and for example, the defective sewing may be detected based on the measurement result of the upstream side tension sensor 233 having the thread supplying portion 23 that supplies the thread 4 to the sewing machine 21.

The above embodiments are to facilitate understanding of this invention, and should not be interpreted to limit this invention in any way. Further, it is needless to say that this invention may be changed or modified without departing from the scope thereof, and this invention includes its equivalents.

What is claimed is:

1. A manufacturing apparatus of a tampon that has an absorber that absorbs bodily fluids and a removal string to remove the absorber from inside a vagina, the manufacturing apparatus of a tampon comprising:
a sewing machine that sews the absorber and the removal string together; and
a defective sewing detecting portion that detects defective sewing between the absorber and the removal string.

2. A manufacturing apparatus of a tampon according to claim 1, wherein
the defective sewing detecting portion detects the defective sewing in a state where a tension is applied to the removal string along a longitudinal direction of the removal string.

3. A manufacturing apparatus of a tampon according to claim 1, wherein
the defective sewing detecting portion detects the defective sewing based on a tension of a thread of the sewing machine that sews the absorber and the removal string together.

4. A manufacturing apparatus of a tampon according to claim 3, wherein
the defective sewing detecting portion detects the defective sewing by performing at least one of
comparing a maximum value of the tension of the thread within a predetermined period and a first threshold value, and
comparing the tension of the thread and a second threshold value lower than the first threshold value.

5. A manufacturing apparatus of a tampon according to claim 3, wherein
the defective sewing detecting portion detects the defective sewing, based on a tension of an upper thread that has been passed through a sewing needle that moves up and down, of the thread.

6. A manufacturing apparatus of a tampon according to claim 5, wherein
the sewing machine has an upper thread tension regulator that regulates the tension of the upper thread and a thread take up lover that moves up and down in accordance with the up and down movement of the sewing needle, and
the defective sewing detecting portion detects the defective sewing based on the tension of the upper thread that is measured with a tension sensor provided between the upper thread tension regulator and the thread take up lever.

7. A manufacturing apparatus of a tampon according to claim 1, wherein
a tension regulating portion and a speed sensor are provided between the sewing machine and the supply source of the thread of the sewing machine that sews the absorber and the removal string together, the tension regulating portion regulating a tension of the thread fed from a supply source to be a predetermined value, the speed sensor measuring a speed in which the thread fed from the tension regulating portion is supplied to the sewing machine, and
the defective sewing detecting portion detects the defective sewing based on a measurement result of the speed sensor.

8. A manufacturing method of a tampon that has an absorber that absorbs bodily fluids and a removal string to remove the absorber from inside a vagina, the manufacturing method of a tampon comprising:
sewing the absorber and the removal string together; and
detecting defective sewing between the absorber and the removal string.
